(19) United States
(12) Patent Application Publication
(10) Pub. No.: US 2026/0240546 A1
(43) Pub. Date: Aug. 20, 2026

(54) DELIVERY CATHETERS, VASCULAR IMPLANTATION ASSEMBLIES, AND METHODS OF USE

(71) Applicant: BARD PERIPHERAL VASCULAR, INC., Franklin Lakes, NJ (US)

(72) Inventors: Danielle ELDRED, Tempe, AZ (US); Keith S. HARRIS, Mesa, AZ (US); Hannah HORECZKO, Tempe, AZ (US); Sanya Vipul MEHTA, Phoenix, AZ (US); Gabriella ROMANO, Tempe, AZ (US)

(73) Assignee: BARD PERIPHERAL VASCULAR, INC., Franklin Lakes, NJ (US)

(21) Appl. No.: 19/161,849

(22) PCT Filed: Mar. 7, 2023
(86) PCT No.: PCT/US2023/063847

§ 371 Date: Sep. 3, 2025

(51) Int. Cl. A61B 17/12; A61B 17/00

(52) U.S. Cl. CPC A61B 17/12109; A61B 17/00234; A61B 2017/00305; A61B 2017/00367; A61B 2017/12054

(57) ABSTRACT

Delivery catheters (116), vascular implantation assemblies (100), and methods of use to implant a vascular implant (136) comprising a proximal end (135) may include the delivery catheter including a catheter body defining an implantation window (138) and a toggle switch assembly (120) coupled to the catheter body and moveable between an expanded position, blocking the implantation window, and a compressed position allowing the vascular implant to move through the implantation window. The toggle switch assembly includes a spring (122) and a blocking device (128) mounted to the spring, wherein the spring is compressible to move the toggle switch assembly to the compressed position to release the vascular implant from the delivery catheter.

### FIELD

The present disclosure generally relates to delivery catheters and methods of use and, more specifically, to delivery catheters, vascular implantation assemblies including such delivery catheters, and methods of use to deploy a vascular implant in a blood vessel.

### BACKGROUND

Medical procedures such as vascular embolization place a tube such as a catheter into a blood vessel, either an artery or a vein, and the catheter is advanced through the blood vessels to a position at which embolization agents are introduced. Afterwards, the catheter is removed. A need exists for improved mechanisms of deployment for devices such as vascular embolization devices to introduce such embolization agents at treatment sites in a blood vessel.

### SUMMARY

In one embodiment, the vascular implantation assembly comprises a vascular implant with a proximal end and a delivery catheter. The delivery catheter includes a catheter body and a toggle switch assembly. The catheter body defines an implantation window. The toggle switch assembly is coupled to the catheter body and moveable between an expanded position, blocking the implantation window, and a compressed position allowing the vascular implant to move through the implantation window. The toggle switch assembly includes a spring and a blocking device mounted to the spring. The spring is compressible to move the toggle switch assembly to the compressed position to release the vascular implant from the delivery catheter.

In another embodiment, a delivery catheter for delivery of a vascular implant comprises a catheter body defining an implantation window and a toggle switch assembly coupled to the catheter body and moveable between an expanded position, blocking the implantation window, and a compressed position allowing the vascular implant to move through the implantation window. The toggle switch assembly includes a spring and a blocking device mounted to the spring, wherein the spring is compressible to move the toggle switch assembly to the compressed position to release the vascular implant from the delivery catheter.

In yet another embodiment, a method of implanting a vascular implant comprises advancing a vascular implantation assembly to a target position within a vessel. The vascular implantation assembly comprises a vascular implant and a delivery catheter releasably coupled to the vascular implant via a toggle switch assembly. The toggle switch assembly comprises a spring and a blocking device mounted to the spring. The method further comprises compressing the spring to move the toggle switch assembly from an expanded position blocking an implantation window formed in the delivery catheter to a compressed position. Movement to the compressed position moves the blocking device and causes the vascular implant to advance through the implantation window and implant at the target position.

These and additional features provided by the embodiments described herein will be more fully understood in view of the following detailed description, in conjunction with the drawings

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a vascular implantation assembly including an in-line pull spring release mechanism, such that a spring longitudinal axis is coaxially aligned with an assembly connector of a toggle switch assembly, and disposed in a first position at a target site in a vessel, with a spring of the toggle switch assembly of the vascular implantation assembly in a naturally biased, relaxed position, according to one or more embodiments shown and described herein;

FIG. 2 schematically depicts the vascular implantation assembly of FIG. 1 in a second position with an outer shaft of the vascular implantation assembly retracted to expose a vascular implant;

FIG. 3 schematically depicts the vascular implantation assembly of FIG. 2 in a third position with the toggle switch assembly of the vascular implantation assembly retracted such that the spring is in a compressed position and a previously blocked implantation window is exposed to allow for release of the vascular implant;

FIG. 4 schematically depicts the vascular implantation assembly of FIG. 3 in a fourth position with a delivery catheter as an inner shaft of the vascular implantation assembly retracted and the vascular implant released;

FIG. 5 schematically depicts another embodiment of a vascular implantation assembly including an in-line push spring release mechanism and disposed in a first position at a target site in a vessel, with a spring of a toggle switch assembly of the vascular implantation assembly in a naturally biased, relaxed position, according to one or more embodiments shown and described herein;

FIG. 6 schematically depicts the vascular implantation assembly of FIG. 5 in a second position with an outer shaft and delivery catheter advance to exit an introducer sheath of the vascular implantation assembly;

FIG. 7 schematically depicts the vascular implantation assembly of FIG. 6 in a third position with the outer shaft of the vascular implantation assembly retracted to expose a vascular implant;

FIG. 8 schematically depicts the vascular implantation assembly of FIG. 7 in a fourth position with the toggle switch assembly of the vascular implantation assembly distally pushed forward such that the spring is in a compressed position and a previously blocked implantation window is exposed to allow for release of the vascular implant;

FIG. 9 schematically depicts the vascular implantation assembly of FIG. 8 in a fifth position with a delivery catheter as an inner shaft of the vascular implantation assembly retracted and the vascular implant released;

FIG. 10 schematically depicts another vascular implantation assembly including an angled spring release mechanism, such that a spring longitudinal axis is angled with respect to an assembly connector of a toggle switch assembly, and disposed in a first position at a target site in a vessel, with a spring of the toggle switch assembly of the vascular implantation assembly in a naturally biased, relaxed position, according to one or more embodiments shown and described herein;

FIG. 11 schematically depicts the vascular implantation assembly of FIG. 10 in a second position with an outer shaft of the vascular implantation assembly retracted to expose a vascular implant;

FIG. 12 schematically depicts the vascular implantation assembly of FIG. 11 in a third position with the toggle switch assembly of the vascular implantation assembly retracted such that the spring is in a compressed position and a previously blocked implantation window is exposed to allow for release of the vascular implant;

FIG. 13 schematically depicts the vascular implantation assembly of FIG. 12 in a fourth position with a delivery catheter as an inner shaft of the vascular implantation assembly retracted and the vascular implant released;

FIG. 14 schematically depicts a toggle switch of a toggle switch assembly, according to one or more embodiments shown and described herein; and

FIG. 15 depicts a flow diagram for implanting a vascular implant with a vascular implantation assembly, according to one or more embodiments shown and described herein.

### DESCRIPTION

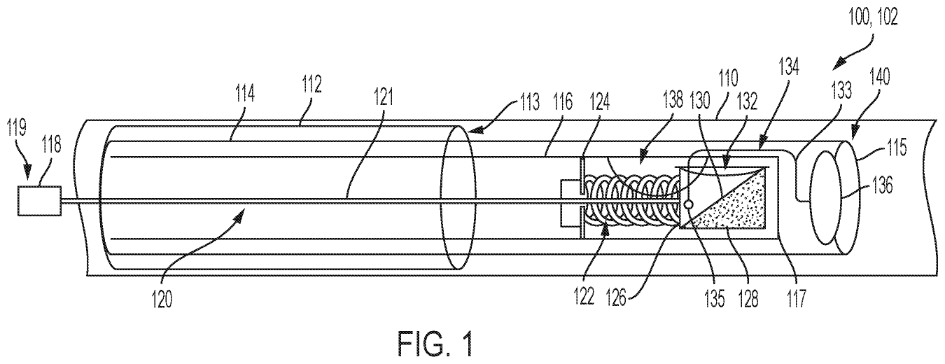

Reference will now be made in detail to embodiments of delivery catheters, vascular implantation assemblies, and methods of use to implant a vascular implant, examples of which are illustrated in the accompanying drawings. Whenever possible, the same reference numerals will be used throughout the drawings to refer to the same or like parts. One embodiment of a vascular implantation assembly 100 including a delivery catheter 116 is depicted in FIG. 1. Various embodiments of delivery catheters, vascular implantation assemblies, and methods of use to implant a vascular implant will be described in further detail herein with specific reference to the appended drawings.

Referring to FIGS. 1-4, an embodiment of a vascular implantation assembly 100 is shown. The vascular implantation assembly 100 of FIGS. 1-4 as described herein includes an in-line pull spring release mechanism, in which a longitudinal axis of a spring 122 is coaxially aligned with an assembly connector 121 of a toggle switch assembly 120 of the vascular implantation assembly 100 and is configured to be pulled via retraction in a proximal direction.

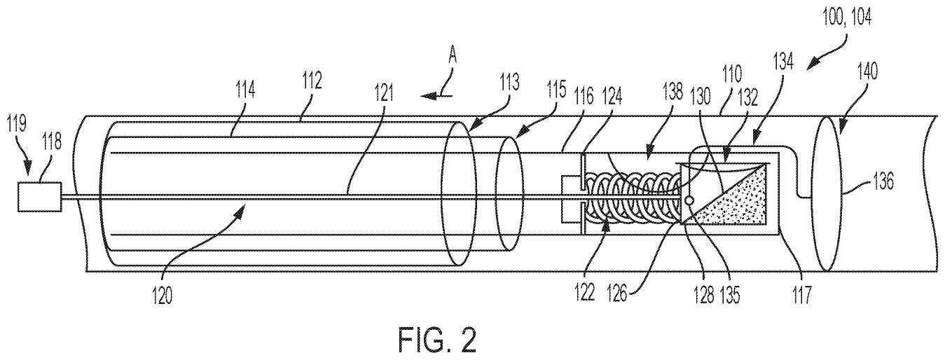

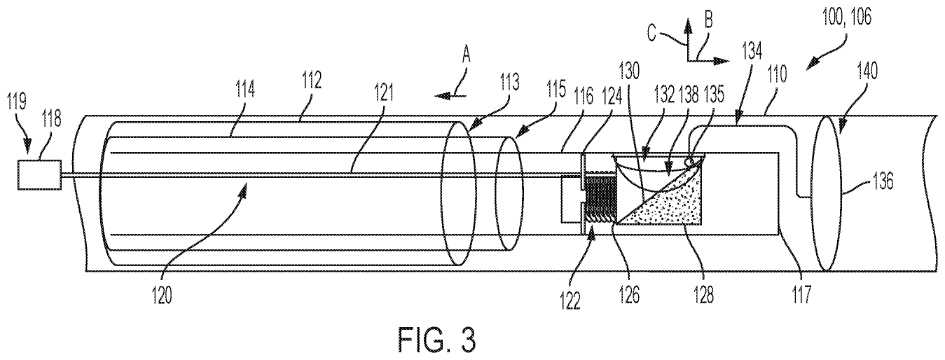

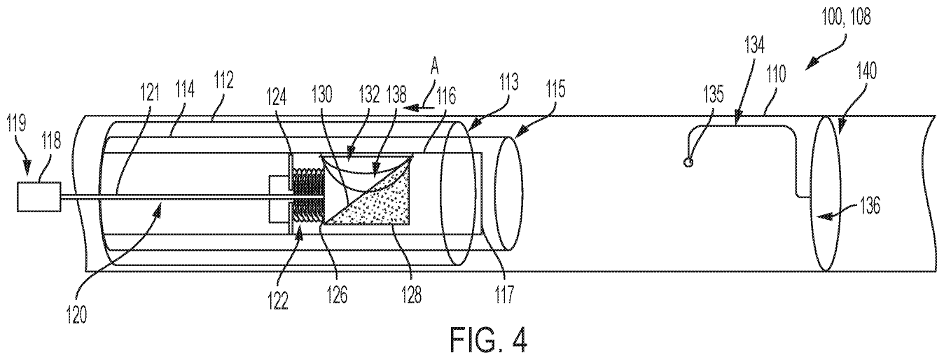

Referring to FIG. 1, the vascular implantation assembly 100 is disposed in a first position 102 at a target site 140 in a vessel 110. In the first position 102, the spring 122 of the toggle switch assembly 120 of the vascular implantation assembly 100 is in a naturally biased, relaxed position. The vascular implantation assembly 100 includes a vascular implant 136 including a proximal end 135 and a delivery catheter 116 as an inner shaft including a catheter body. An implant assembly 134 includes the proximal end 135 as an anchor connected to the vascular implant 136, such as via a connecting line 133. The catheter body defines an implantation window 138. The delivery catheter 116 further includes the toggle switch assembly 120 coupled to the catheter body and moveable between an expanded position, blocking the implantation window 138 (FIGS. 1-2), and a compressed position allowing the vascular implant 136 to move through the implantation window 138 (FIGS. 3-4). In embodiments, the toggle switch assembly 120 is positioned within a lumen of the catheter body of a delivery catheter 116.

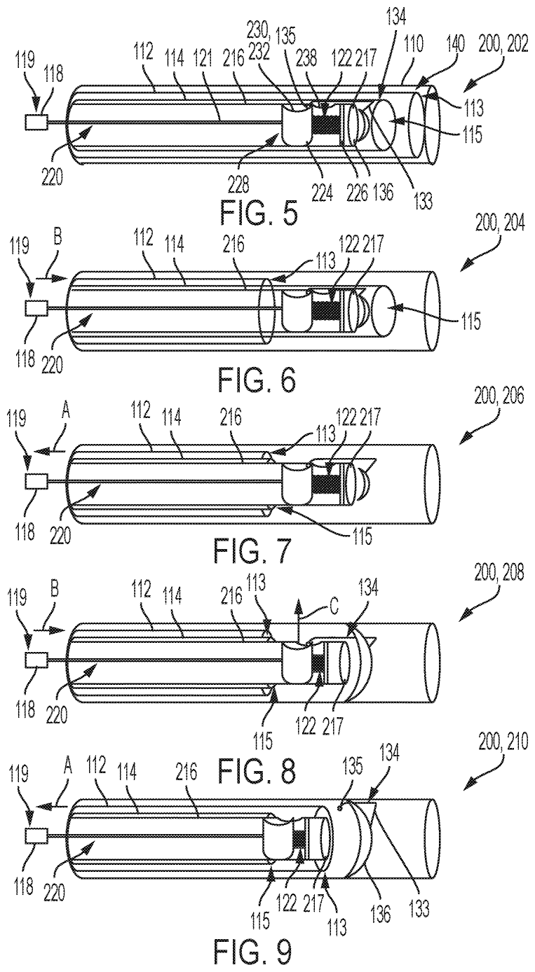

The toggle switch assembly 120 includes the spring 122 and a blocking device 128 mounted to the spring 122. The spring 122 is configured to be compressible to move the toggle switch assembly 120 to the compressed position to release the vascular implant 136 from the delivery catheter 116, as shown in FIGS. 3-4. As shown in FIGS. 1-4, and similarly in FIGS. 5-9 described in greater detail below, the spring 122 comprises a compression axis positioned parallel to a longitudinal axis of the catheter body of the delivery catheter 116.

Referring again to FIG. 1, the blocking device 128 includes a ramped surface 130 configured to advance the proximal end 135 of the vascular implant 136 through the implantation window 138 as the toggle switch assembly 120 is moved to the compressed position of FIGS. 3-4. The blocking device 128 is configured to impinge the proximal end 135 of the vascular implant 136 between the blocking device 128 and the catheter body of the delivery catheter 116 when the toggle switch assembly 120 is in the expanded position of FIGS. 1-2.

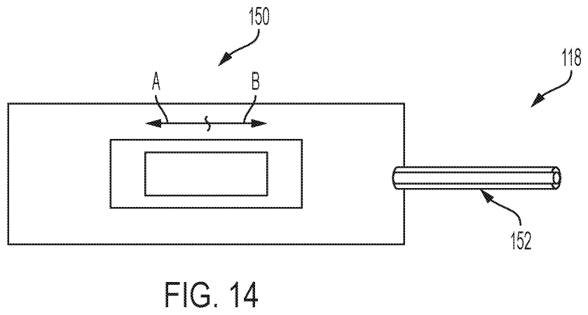

The catheter body of the delivery catheter 116 further includes one or more spring-interface ledges, such as spring-interface proximal stop 124, engaged with the spring 122 and configured to limit a displacement of the spring 122 in a proximal direction of arrow A in FIG. 2. The spring 122 is disposed between the spring-interface proximal stop 124 and a spring-interface distal stop 126 on the blocking device 128. The vascular implantation assembly 100 may further include a handle, such as including a toggle switch 118 of the toggle switch assembly as described in greater detail below with respect to FIG. 14, the handle coupled to the delivery catheter 116 and including a user input coupled to the toggle switch assembly 120. Operation of the user input may move the toggle switch assembly from the expanded position of FIGS. 1-2 to the compressed position of FIGS. 3-4.

Referring again to FIG. 1, the vascular implantation assembly 100 has been introduced into the vessel 110 until reaching the target site 140 of and within the vessel 110, which is a location at which to deploy the vascular implant 136. The vascular implantation assembly 100 further includes an introducer sheath 112 and an outer shaft 114. The introducer sheath 112 defines an opening 113 at a distal end sized and shaped to be disposed about and longitudinally be slidably displaced with respect to the outer shaft 114. The outer shaft 114 defines an opening 115 at a distal end sized and shaped to be disposed about and longitudinally be slidably displaced with respect to the delivery catheter 116. The body of the delivery catheter 116 defines the implantation window 138.

With respect to the toggle switch assembly 120, the toggle switch 118 is disposed at a proximal end 119 and attaching to the blocking device 128 via an assembly connector 121. In embodiments, the assembly connector may be a wire or other connecting component. The blocking device 128 includes a ramped surface 130 and a spring stop window 132. Portions of the implant assembly 134 such as the implant proximal end 135 are disposed and impinged within the blocking device 128 in the first position 102.

In the first position 102, the introducer sheath 112 is shown in a proximal position with respect to the outer shaft 114 and the delivery catheter 116 that includes the distal end disposed within the outer shaft 114. Thus, the outer shaft 114 and the delivery catheter 116 are shown as having been pushed distally from to exit the introducer sheath 112. The spring 122 is in a relaxed, naturally biased position, and the implant proximal end 135 of the implant assembly 134 is disposed and impinged within the blocking device 128 via the spring stop window 132 that is closed by the body of the delivery catheter 116 distal to the implantation window 138 and the body of the outer shaft 114 proximal to the opening 115.

Referring to FIG. 2, a second position 104 is shown in which the outer shaft 114 has been retracted in the proximal direction of arrow A to expose the vascular implant 136. The retraction of the outer shaft 114 exposes the implantation window 138 and the vascular implant 136. In embodiments, the vascular implant 136 may then expand within the vessel 110, as shown in FIG. 2.

Referring to FIG. 3, a third position 106 is shown in in which the toggle switch assembly 120 is retracted in the proximal direction of arrow A. Thus, the spring 122 is in a compressed position between the spring-interface proximal stop 124 and the spring-interface distal stop 126, and the implantation window 138 of the delivery catheter 116 previously blocked by the blocking device 128 is exposed and aligned with the spring stop window 132 to allow for release of the vascular implant 136 from the blocking device 128. By way of example, and not as a limitation, the implant proximal end 135 distally rides up the ramped surface 130 such as in a distal direction of arrow B until reaching a joint opening formed by the aligned spring stop window 132 and the implantation window 138. At this point, the implant proximal end 135 may be released from the joint opening in the direction of arrow C.

Referring to FIG. 4, a fourth position 108 is shown in which the implant proximal end 135 has been released through the joint opening and the vascular assembly 134 is disposed within the vessel 110. Further, the delivery catheter 116 has been retracted in the proximal direction of arrow A into the outer shaft 114 such that the distal end 117 of the delivery catheter 116 is proximally disposed within the outer shaft 114 with respect to the opening 115 of the outer shaft 114. The vascular assembly 134 is thus released and distally spaced from the delivery catheter 116.

Referring to FIGS. 5-9, an embodiment of a vascular implantation assembly 200 is shown. The vascular implantation assembly 200 of FIGS. 5-9 as described herein includes an in-line push spring release mechanism, in which the spring longitudinal axis of a spring 122 is coaxially aligned with an assembly connector 121 of a toggle switch assembly 220 of the vascular implantation assembly 200 and is configured to be coaxially pushed in a distal direction.

Referring to FIG. 5, the vascular implantation assembly 200 is disposed in a first position 202 at the target site 140 in the vessel 110. In the first position 202, the spring 122 of the toggle switch assembly 220 of the vascular implantation assembly 200 is in a naturally biased, relaxed position. The vascular implantation assembly 200 includes a vascular implant 136 including a proximal end 135 and a delivery catheter 216 as an inner shaft including a catheter body and having a distal end 217. The implant assembly 134 may include the proximal end 135 as an anchor, as a non-limiting example, connected to the vascular implant 136, such as via a connecting line 133. The catheter body of the delivery catheter 216 defines an implantation window 238. The delivery catheter 216 further includes the toggle switch assembly 220 coupled to the catheter body and moveable between an expanded position, blocking the implantation window 238 (FIGS. 5-7), and a compressed position allowing the vascular implant 136 to move through the implantation window 238 (FIGS. 8-9), each of which FIGS. 5-7 will be described separately below in greater detail. Prior to movement of the toggle switch assembly 220 to the compressed position of FIG. 8-9, an outer shaft 114 and the delivery catheter 216 may be advanced from the introducer sheath 112 in a distal direction of arrow B as shown in FIG. 6. The outer shaft 114 that is disposed about a distal end of the delivery catheter 216 in FIG. 6 may be retracted in a proximal direction of arrow A as shown in FIG. 7 to expose the vascular implant 136 in the vessel 110. The retraction of the outer shaft 114 exposes the distal end of the delivery catheter 216 such that the exposed vascular implant 136 expands within the vessel 110 prior to compression of the spring 122 as shown in FIG. 8 to move the toggle switch assembly to the compressed position of FIGS. 8-9. In embodiments, the toggle switch assembly 220 is positioned within a lumen of the catheter body of a delivery catheter 216.

The toggle switch assembly 220 includes the spring 122 and a blocking device 228 mounted to the spring 122. The spring 122 is compressible such that the spring 122 is configured to be compressed in the distal direction of arrow B (FIG. 8) to move the toggle switch assembly 220 to the compressed position to release the vascular implant 136 from the delivery catheter 216, as shown in FIGS. 8-9. Thus, the spring 122 comprises a compression axis positioned parallel to a longitudinal axis of the catheter body of the delivery catheter 216.

Referring again to FIG. 5, the blocking device 228 includes a spring stop window 232 defining a ramped surface configured to advance the proximal end 135 of the vascular implant 136 through the implantation window 238 as the toggle switch assembly 220 is moved to the compressed position of FIGS. 8-9. The blocking device 228 is configured to impinge the proximal end 135 of the vascular implant 136 between the blocking device 228 and the catheter body of the delivery catheter 216 when the toggle switch assembly 220 is in the expanded position of FIGS. 5-7.

The catheter body of the delivery catheter 216 further includes one or more spring-interface ledges, such as spring-interface proximal stop 224, engaged with the spring 122 and configured to limit a displacement of the spring 122 in a proximal direction of arrow A in FIG. 7. The spring 122 is disposed between the spring-interface proximal stop 224 on the blocking device 228 and a spring-interface distal stop 226. The vascular implantation assembly 200 may further include a handle, such as including a toggle switch 118 of the toggle switch assembly as described in greater detail below with respect to FIG. 14, the handle coupled to the delivery catheter 116 and including a user input coupled to the toggle switch assembly 120. Operation of the user input may move the toggle switch assembly from the expanded position of FIGS. 5-7 to the compressed position of FIGS. 8-9.

Referring again to FIG. 5, the vascular implantation assembly 200 has been introduced into the vessel 110 until reaching the target site 140 of and within the vessel 110, which is a location at which to deploy the vascular implant 136. The vascular implantation assembly 200 further includes the introducer sheath 112 and the outer shaft 114, similar to as described for the vascular implantation assembly 110 above. The outer shaft 114 defines the opening 115 at a distal end sized and shaped to be disposed about and longitudinally be slidably displaced with respect to the delivery catheter 216.

With respect to the toggle switch assembly 220, the toggle switch 118 is disposed at a proximal end 119 and attaching to the blocking device 228 via an assembly connector 121. The blocking device 228 includes a ramped surface 230 and a spring stop window 232. Portions of the implant assembly 134 such as the implant proximal end 135 are disposed and impinged within the blocking device 228 in the first position 202.

In the first position 202, the introducer sheath 112 is shown in a distal position with respect to the outer shaft 114 and the delivery catheter 116 that includes the distal end disposed within the outer shaft 114 such that the opening 113 of the introducer sheath 112 is disposed distally past the opening 113 of the outer shaft 114. The spring 122 is in a relaxed, naturally biased position, and the implant proximal end 135 of the implant assembly 134 is disposed and impinged within the blocking device 228 via the spring stop window 232 that is closed by the body of the delivery catheter 216 proximal to the implantation window 238 and the body of the outer shaft 114 proximal to the opening 115.

Referring to FIG. 6, the vascular implantation assembly 200 is shown in the second position 204 with the introducer sheath 112 of the vascular implantation assembly is either retracted, or the outer shaft 114 and the delivery catheter 216 distally advanced out of the opening 113 of the introducer sheath 112, to expose a vascular implant 136. Thus, the outer shaft 114 and the delivery catheter 216 are shown as having exited the introducer sheath 112. The spring 122 is in a relaxed, naturally biased position, and the implant proximal end 135 of the implant assembly 134 is disposed and impinged within the blocking device 228 via the spring stop window 232 that is closed by the body of the delivery catheter 216 distal to the implantation window 238 and the body of the outer shaft 114 proximal to the opening 115

Referring to FIG. 7, the vascular implantation assembly 200 is shown in a third position 206 where the outer shaft 114 is retracted in the proximal direction of arrow A to expose the distal end 217 of the delivery catheter 216 and the vascular implant 136. The retraction of the outer shaft 114 exposes the implantation window 238 and the vascular implant 136. In embodiments, the vascular implant 136 may then expand within the vessel 110, as shown in FIG. 8.

Referring to FIG. 8, the vascular implantation assembly 200 is shown in a fourth position 208 with the toggle switch assembly 220 of the vascular implantation assembly 200 distally pushed forward in the distal direction of arrow B such that the spring 122 is in a compressed position and a previously blocked spring stop window 232 is exposed to allow for release of the vascular implant 136 from the delivery catheter 116. Thus, the spring 122 is in a compressed position between the spring-interface proximal stop 224 and the spring-interface distal stop 226, and the spring stop window 232 of the delivery catheter 216 previously blocked by the blocking device 228 is exposed and aligned with the implantation window 238 to allow for release of the vascular implant 136 from the blocking device 228. By way of example, and not as a limitation, the implant proximal end 135 distally rides up the ramped surface such as in a distal direction of arrow B until at or is distally positioned at a joint opening formed by the aligned spring stop window 232 and the implantation window 238. At this point, the implant proximal end 135 may be released from the joint opening in the direction of arrow C.

Referring to FIG. 9, the vascular implantation assembly 200 is shown in a fifth position 210 with the delivery catheter 216 retracted and the vascular implant 136 released from the delivery catheter 216. The implant proximal end 135 has been released through the joint opening and the vascular assembly 134 is disposed within the vessel 110. Further, the delivery catheter 216 has been retracted in the proximal direction of arrow A into the outer shaft 114 such that the distal end 217 of the delivery catheter 216 is proximally disposed within the outer shaft 114 with respect to the opening 115 of the outer shaft 114. The vascular assembly 134 is thus released and distally spaced from the delivery catheter 216.

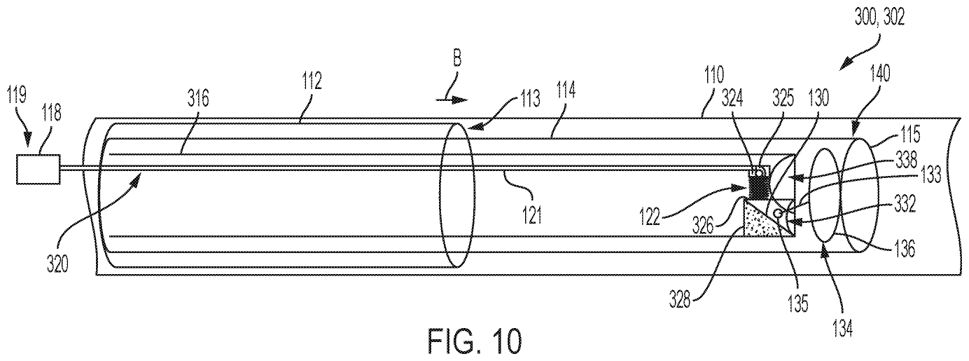

Referring to FIGS. 10-13, an embodiment of a vascular implantation assembly 300 is shown. The vascular implantation assembly 300 includes an angled spring release mechanism, such that the spring longitudinal axis of the spring 122 is angled with respect to the assembly connector 121 of a toggle switch assembly 320. In embodiments, the spring longitudinal axis of the spring 122 may be angled 90 degrees with respect to, and thus be perpendicular to, the assembly connector 121 of the toggle switch assembly 320.

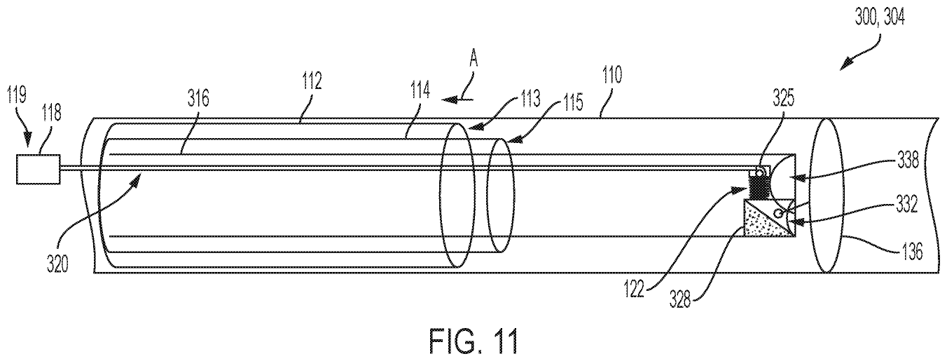

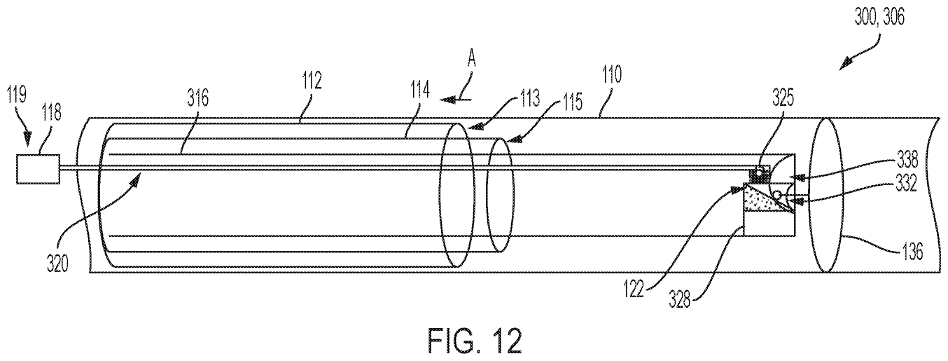

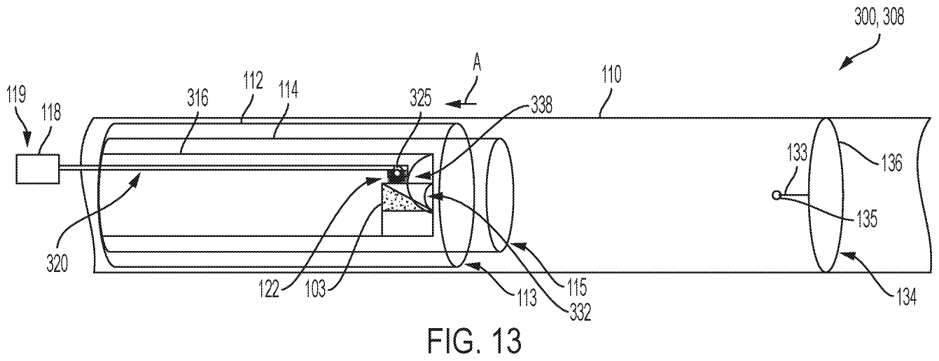

FIG. 10 depicts the vascular implantation assembly 300 disposed in a first position 302 at the target site 140 in the vessel 110. In the first position 302, the spring 122 of the toggle switch assembly 320 of the vascular implantation assembly 300 is in a naturally biased, relaxed position. The vascular implantation assembly 300 includes the vascular implant 136 including a proximal end 135 and a delivery catheter 316 as an inner shaft including a catheter body. The implant assembly 134 includes the proximal end 135 as an anchor connected to the vascular implant 136, such as via a connecting line 133. The catheter body defines an implantation window 338 that is disposed in a distal-facing direction. The delivery catheter 316 further includes the toggle switch assembly 320 coupled to the catheter body and moveable between an expanded position, blocking the implantation window 338 (FIGS. 10-11), and a compressed position allowing the vascular implant 136 to move through the implantation window 338 (FIGS. 12-13). In embodiments, the toggle switch assembly 320 is positioned within a lumen of the catheter body of a delivery catheter 316.

The toggle switch assembly 320 includes the spring 122 and a blocking device 328 mounted to the spring 122. The spring 122 is configured to be compressed to move the toggle switch assembly 320 to the compressed position to release the vascular implant 136 from the delivery catheter 316, as shown in FIGS. 12-13. As shown in FIGS. 10-13, the spring 122 comprises a compression axis positioned at an angle such as perpendicular to a longitudinal axis of the catheter body of the delivery catheter 316.

Referring again to FIG. 10, the blocking device 328 includes the ramped surface 130 configured to advance the proximal end 135 of the vascular implant 136 through the implantation window 338 as the toggle switch assembly 320 is moved to the compressed position of FIGS. 12-13. The blocking device 328 is configured to impinge the proximal end 135 of the vascular implant 136 between the blocking device 328 and the catheter body of the delivery catheter 316 when the toggle switch assembly 320 is in the expanded position of FIGS. 10-11.

The catheter body of the delivery catheter 316 further includes one or more spring-interface ledges. The spring 122 is disposed between the spring-interface stop 324 and a spring-interface stop 326 on the blocking device 128. The vascular implantation assembly 300 may further include a handle, such as including a toggle switch 118 of the toggle switch assembly as described in greater detail below with respect to FIG. 14, the handle coupled to the delivery catheter 316 and including a user input coupled to the toggle switch assembly 320. Operation of the user input may move the toggle switch assembly from the expanded position of FIGS. 10-11 to the compressed position of FIGS. 12-13.

Referring again to FIG. 10, the vascular implantation assembly 300 has been introduced into the vessel 110 until and has reached the target site 140 of and within the vessel 110, which is a location at which to deploy the vascular implant 136. The outer shaft 114 defines an opening 115 at a distal end sized and shaped to be disposed about and longitudinally be slidably displaced with respect to the delivery catheter 316. The body of the delivery catheter 316 defines the implantation window 338 at a distal end, which implantation window 338 is distal-facing.

With respect to the toggle switch assembly 320, the toggle switch 118 is disposed at a proximal end 119 and attaching to the blocking device 328 via an assembly connector 121 and a pulley 325. The blocking device 328 includes the ramped surface 130 and the spring stop window 332. Portions of the implant assembly 134 such as the implant proximal end 135 are impinged within the blocking device 328 in the first position 302.

In the first position 302, the introducer sheath 112 is shown in a proximal position with respect to the outer shaft 114 and the delivery catheter 316 that includes the distal end disposed within the outer shaft 114. Thus, the outer shaft 114 and the delivery catheter 316 are shown as having exited the introducer sheath 112. The spring 122 is in a relaxed, naturally biased position, and the implant proximal end 135 of the implant assembly 134 is disposed and impinged within the blocking device 328 via the spring stop window 332 that is closed by the body of the delivery catheter 116 below the implantation window 338.

Referring to FIG. 11, the vascular implantation assembly 300 is shown in a second position 304 where outer shaft 114 is retracted in the proximal direction of arrow A, exposing and the vascular implant 136. The retraction of the outer shaft 114 exposes the vascular implant 136. In embodiments, the vascular implant 136 may then expand within the vessel 110, as shown in FIG. 11. Referring to FIG. 12, the vascular implantation assembly 300 is shown in a third position 306 where the toggle switch assembly 320 is retracted in the proximal direction of arrow A to pull the assembly connector 121 over pulley 325, which compresses the spring 122, and which exposes the spring stop window 332. Thus, the spring 122 is in a compressed position between the spring-interface stop 324 and the spring-interface stop 326, and the spring stop window 332 of previously blocked by the body of the delivery catheter 116 is exposed and aligned with the implantation window 338 to allow for release of the vascular implant 136 from the blocking device 328. By way of example, and not as a limitation, the implant proximal end 135 distally rides up the ramped surface 130 until at a joint opening formed by the aligned spring stop window 332 and the implantation window 338. At this point, the implant proximal end 135 may be released from the joint opening in the distal direction (e.g., opposite the proximal direction of arrow A).

Referring to FIG. 13, the vascular implantation assembly 300 is shown in a fourth position 308 in which the delivery catheter 316 is retracted and the vascular implant 136 is released. In the fourth position 308, the implant proximal end 135 has been released through the joint opening and the vascular assembly 134 is disposed within the vessel 110. Further, the delivery catheter 316 has been retracted in the proximal direction of arrow A into the outer shaft 114 such that the distal end of the delivery catheter 116 is proximally disposed within the outer shaft 114 with respect to the opening 115 of the outer shaft 114. The vascular assembly 134 is thus released and distally spaced from the delivery catheter 316.

FIG. 14 shows the toggle switch 118, which may be of any toggle switch assembly 120, 220, 320 described herein. Toggle switch 118 has toggle switch proximal end 150 and a toggle switch distal end 152. Toggle switch 118 is configured to be moved in the proximal direction of arrow A towards the toggle switch proximal end 150 or in distal direction of arrow B towards the toggle switch distal end 152. Toggle Switch 118 may also be disposed within a handle and operated with the vascular implantation assemblies 100, 200, 300 as described herein. In embodiments, the handle may include one or more attachment mechanisms such as a luer attachment to allow for flushing between the inner and outer catheters as described herein if and as needed and/or to introduce saline.

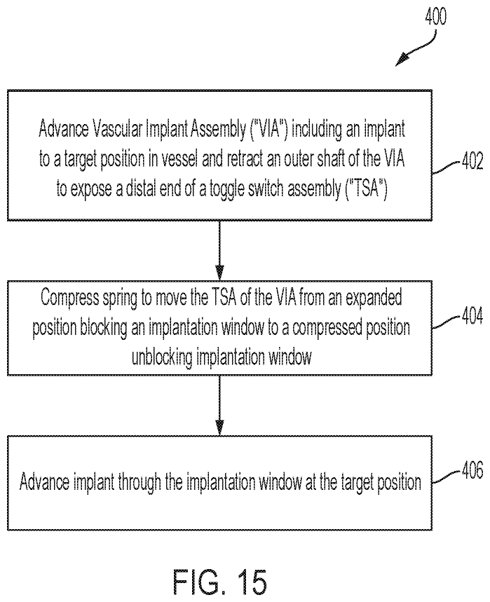

FIG. 15 depicts a method 400 of implanting the vascular implant 136 as described herein. In block 402, a vascular implant assembly (“VIA”) as one of vascular implantation assemblies 100, 200, 300 including the implant 136 is advanced to the target position as target site 140 in the vessel 110 as described herein. The vascular implantation assembly 100, 200, 300, includes the vascular implant 136 and a respective delivery catheter 116, 216, 316, releasably coupled to the vascular implant 136 via a respective toggle switch assembly 120, 220, 320. The toggle switch assembly 120, 220, 320 includes a spring 122 and a respective blocking device 128, 228, 328 mounted to the spring 122 as described herein. An outer shaft 114 of the VIA is retracted to expose a distal end of the toggle switch assembly 120, 220, 320. Prior to retraction, the outer shaft 114 is disposed about the distal end of the toggle switch assembly 120, 220, 320 (within a distal end of the delivery catheter 116, 216,316). Retraction of the outer shaft 114 in a proximal direction to expose the distal end of the toggle switch assembly 120, 220, 320 (and the delivery catheter 116, 216, 316) allows for an expansion of the vascular implant 136, a portion of which is disposed and impinged within the delivery catheter 116, 216, 316, within the vessel 110 prior to compressing the spring 122.

In block 404, the spring 122 is compressed to move the toggle switch assembly 120, 220, 320 from an expanded position blocking a respective implantation window 138, 238, 338 formed in the respective delivery catheter 116, 216, 316 to a compressed position. In embodiments, as shown in FIGS. 1-4 for the vascular implantation assembly 100, and as shown in FIGS. 5-9 for the vascular implantation assembly 200, the spring 122 is compressed along a compression axis parallel to a longitudinal axis of the delivery catheter 116, 216. Alternatively, as shown in FIGS. 10-13 for the vascular implantation assembly 300, the spring 122 is compressed along a compression axis angled to, such as perpendicular to, a longitudinal axis of the delivery catheter 316. In embodiments, a user input may be actuated to cause compression of the spring 122.

Movement to the compressed position move the blocking device 128, 228, 328 and causes the vascular implant 136 to eject through the implantation window 138, 238, 338. Thus, in block 406, the implant 136 is advanced from the respective implantation window 138, 238, 338, aligned with a respective spring stop window 132, 232, 332 to form a joint opening with the respective implantation window 138, 238, 338 to advance the implant assembly 134 including the implant 136 at the target site 140. In embodiments, the blocking device 128, 228, 328 may include a ramped surface 130, 230 configured to advance a proximal end (e.g., the implant proximal end 135) of the vascular implant 136 through the implantation window 138, 238, 338 as the toggle switch assembly 120, 220, 320 is moved to the compressed position.

Although particular embodiments have been disclosed herein in detail, this has been done by way of example for purposes of illustration only, and is not intended to be limiting with respect to the scope of the appended claims, which follow. In particular, it is contemplated that various substitutions, alterations, and modifications may be made without departing from the spirit and scope of the disclosure as defined by the claims. Other aspects, advantages, and modifications are considered to be within the scope of the following claims. The claims presented are representative of the embodiments disclosed herein.

### Aspects Listing

Aspect 1. A vascular implantation assembly comprising a vascular implant comprising a proximal end and a delivery catheter. The delivery catheter comprises a catheter body defining an implantation window and a toggle switch assembly coupled to the catheter body. The toggle switch assembly is moveable between an expanded position, blocking the implantation window, and a compressed position allowing the vascular implant to move through the implantation window. The toggle switch assembly comprises a spring and a blocking device mounted to the spring. The spring is compressible to move the toggle switch assembly to the compressed position to release the vascular implant from the delivery catheter.

Aspect 2. The vascular implantation assembly of Aspect 1, wherein the spring comprises a compression axis positioned parallel to a longitudinal axis of the catheter body.

Aspect 3. The vascular implantation assembly of Aspect 1, wherein the spring comprises a compression axis positioned perpendicular to a longitudinal axis of the catheter body.

Aspect 4. The vascular implantation assembly of any of Aspect 1 to Aspect 3, wherein the blocking device comprises a ramped surface configured to advance the proximal end of the vascular implant through the implantation window as the toggle switch assembly is moved to the compressed position.

Aspect 5. The vascular implantation assembly of any of Aspect 1 to Aspect 4, wherein the blocking device is configured to impinge the proximal end of the vascular implant between the blocking device and the catheter body when the toggle switch assembly is in the expanded position.

Aspect 6. The vascular implantation assembly of any of Aspect 1 to Aspect 5, wherein the catheter body comprises one or more spring-interface ledges engaged with the spring and configured to limit a displacement of the spring in a proximal direction.

Aspect 7. The vascular implantation assembly of any of Aspect 1 to Aspect 6, further comprising a handle coupled to the delivery catheter and comprising a user input coupled to the toggle switch assembly, wherein operation of the user input moves the toggle switch assembly from the expanded position to the compressed position.

Aspect 8. A delivery catheter for delivery of a vascular implant comprises a catheter body defining an implantation window and a toggle switch assembly coupled to the catheter body and moveable between an expanded position, blocking the implantation window, and a compressed position allowing the vascular implant to move through the implantation window. The toggle switch assembly comprises a spring and a blocking device mounted to the spring, wherein the spring is compressible to move the toggle switch assembly to the compressed position to release the vascular implant from the delivery catheter.

Aspect 9. The delivery catheter of Aspect 8, wherein the spring comprises a compression axis positioned parallel to a longitudinal axis of the catheter body.

Aspect 10. The delivery catheter of Aspect 8, wherein the spring comprises a compression axis positioned perpendicular to a longitudinal axis of the catheter body.

Aspect 11. The delivery catheter of any of Aspect 8 to Aspect 10, wherein the blocking device comprises a ramped surface configured to advance a proximal end of the vascular implant through the implantation window as the toggle switch assembly is moved to the compressed position.

Aspect 12. The delivery catheter of any of Aspect 8 to Aspect 11, wherein the blocking device is configured to impinge a proximal end of the vascular implant between the blocking device and the catheter body when the toggle switch assembly is in the expanded position.

Aspect 13. The delivery catheter of any of Aspect 8 to Aspect 12, wherein the catheter body comprises one or more spring-interface ledges engaged with the spring and configured to limit a displacement of the spring in a proximal direction.

Aspect 14. The delivery catheter of any of Aspect 8 to Aspect 13, further comprising a user input coupled to the toggle switch assembly, wherein operation of the user input moves the toggle switch assembly from the expanded position to the compressed position.

Aspect 15. The delivery catheter of any of Aspect 8 to Aspect 14, wherein the toggle switch assembly is positioned within a lumen of the catheter body.

Aspect 16. A method of implanting a vascular implant comprises advancing a vascular implantation assembly to a target position within a vessel. The vascular implantation assembly comprises a vascular implant and a delivery catheter releasably coupled to the vascular implant via a toggle switch assembly, the toggle switch assembly comprising a spring, and a blocking device mounted to the spring. The method further comprises compressing the spring to move the toggle switch assembly from an expanded position blocking an implantation window formed in the delivery catheter to a compressed position, wherein movement to the compressed position moves the blocking device and causes the vascular implant to advance through the implantation window and implant at the target position.

Aspect 17. The method of Aspect 16, wherein compressing the spring comprises compressing the spring along a compression axis one of parallel or perpendicular to a longitudinal axis of the delivery catheter.

Aspect 18. The method of Aspect 16, further comprising retracting an outer shaft disposed about a distal end of the delivery catheter in a proximal direction to expose the distal end of the delivery catheter and expand the vascular implant within the vessel prior to compressing the spring.

Aspect 19. The method of any of Aspect 16 to Aspect 18, further comprising actuating a user input, whereby actuation of the user input causes compressing of the spring.

Aspect 20. The method of any of Aspect 16 to Aspect 19, wherein the blocking device comprises a ramped surface configured to advance a proximal end of the vascular implant through the implantation window as the toggle switch assembly is moved to the compressed position.

1. A vascular implantation assembly comprising:
a vascular implant comprising a proximal end;
a delivery catheter comprising:
a catheter body defining an implantation window; and
a toggle switch assembly coupled to the catheter body and moveable between an expanded position, blocking the implantation window, and a compressed position allowing the vascular implant to move through the implantation window, the toggle switch assembly comprising:
a spring; and
a blocking device mounted to the spring, wherein the spring is compressible to move the toggle switch assembly to the compressed position to release the vascular implant from the delivery catheter.

2. The vascular implantation assembly of claim 1, wherein the spring comprises a compression axis positioned parallel to a longitudinal axis of the catheter body.

3. The vascular implantation assembly of claim 1, wherein the spring comprises a compression axis positioned perpendicular to a longitudinal axis of the catheter body.

4. The vascular implantation assembly of claim 1, wherein the blocking device comprises a ramped surface configured to advance the proximal end of the vascular implant through the implantation window as the toggle switch assembly is moved to the compressed position.

5. The vascular implantation assembly of claim 1, wherein the blocking device is configured to impinge the proximal end of the vascular implant between the blocking device and the catheter body when the toggle switch assembly is in the expanded position.

6. The vascular implantation assembly of claim 1, wherein the catheter body comprises one or more spring-interface ledges engaged with the spring and configured to limit a displacement of the spring in a proximal direction.

7. The vascular implantation assembly of claim 1, further comprising a handle coupled to the delivery catheter and comprising a user input coupled to the toggle switch assembly, wherein operation of the user input moves the toggle switch assembly from the expanded position to the compressed position.

8. A delivery catheter for delivery of a vascular implant, the delivery catheter comprising:
a catheter body defining an implantation window; and
a toggle switch assembly coupled to the catheter body and moveable between an expanded position, blocking the implantation window, and a compressed position allowing the vascular implant to move through the implantation window, the toggle switch assembly comprising:
a spring; and
a blocking device mounted to the spring, wherein the spring is compressible to move the toggle switch assembly to the compressed position to release the vascular implant from the delivery catheter.

9. The delivery catheter of claim 8, wherein the spring comprises a compression axis positioned parallel to a longitudinal axis of the catheter body.

10. The delivery catheter of claim 8, wherein the spring comprises a compression axis positioned perpendicular to a longitudinal axis of the catheter body.

11. The delivery catheter of claim 8, wherein the blocking device comprises a ramped surface configured to advance a proximal end of the vascular implant through the implantation window as the toggle switch assembly is moved to the compressed position.

12. The delivery catheter of claim 8, wherein the blocking device is configured to impinge a proximal end of the vascular implant between the blocking device and the catheter body when the toggle switch assembly is in the expanded position.

13. The delivery catheter of claim 8, wherein the catheter body comprises one or more spring-interface ledges engaged with the spring and configured to limit a displacement of the spring in a proximal direction.

14. The delivery catheter of claim 8, further comprising a user input coupled to the toggle switch assembly, wherein operation of the user input moves the toggle switch assembly from the expanded position to the compressed position.

15. The delivery catheter of claim 8, wherein the toggle switch assembly is positioned within a lumen of the catheter body.

16. A method of implanting a vascular implant comprising:
advancing a vascular implantation assembly to a target position within a vessel, the vascular implantation assembly comprising a vascular implant and a delivery catheter releasably coupled to the vascular implant via a toggle switch assembly, the toggle switch assembly comprising a spring, and a blocking device mounted to the spring; and
compressing the spring to move the toggle switch assembly from an expanded position blocking an implantation window formed in the delivery catheter to a compressed position, wherein movement to the compressed position moves the blocking device and causes the vascular implant to advance through the implantation window and implant at the target position.

17. The method of claim 16, wherein compressing the spring comprises compressing the spring along a compression axis one of parallel or perpendicular to a longitudinal axis of the delivery catheter.

18. The method of claim 16, further comprising:
retracting an outer shaft disposed about a distal end of the delivery catheter in a proximal direction to expose the distal end of the delivery catheter and expand the vascular implant within the vessel prior to compressing the spring.

19. The method of claim 16, further comprising actuating a user input, whereby actuation of the user input causes compressing of the spring.

20. The method of claim 16, wherein the blocking device comprises a ramped surface configured to advance a proximal end of the vascular implant through the implantation window as the toggle switch assembly is moved to the compressed position.
